(19) United States
(12) Patent Application Publication
(10) Pub. No.: US 2009/0259172 A1
(43) Pub. Date: Oct. 15, 2009

(54) OVER TUBE

(76) Inventors: Koji YAMAOKA, Tokyo (JP); Kenji NODA, Tokyo (JP); Masatsugu OYAMA, Tokyo (JP); Kunitoshi HIRAGA, Tokyo (JP)

Correspondence Address: OSTROLENK FABER GERB & SOFFEN, 1180 AVENUE OF THE AMERICAS, NEW YORK, NY, 100368403, US

(21) Appl. No.: 12/099,848

(22) Filed: Apr. 9, 2008

(51) Int. Cl. A61M 13/00

(52) U.S. Cl. 604/26

(57) ABSTRACT

An over tube comprises an over tube body that has an inner diameter fitting over the outer diameter of an insertion part, and is flexible and expandable in the direction of the outer diameter, a gas supply port provided on the base end side of the over tube body, an expansion/contraction part that is formed integrally with the gas supply port, provided to communicate from the base end side to a predetermined position on a distal end side, and can expand in the direction of the outer diameter by injecting a gas from the gas supply port, and at least one channel that is provided between the inner and the outer diameters of the over tube body, and penetrates from the distal end of the over tube through the base end side.

### FIELD OF THE INVENTION

The present invention relates to an over tube.

### BACKGROUND OF THE INVENTION

In recent years, NOTES (Natural Orifice Translumenal Endoscopic Surgery) techniques for making a hole within the stomach or the intestines by inserting a soft endoscope from a natural orifice such as mouth, nose, anus, etc. of a patient, and for performing a surgery by inserting the endoscope into the hole have been proposed as techniques for reducing the burden on the patient at the time of an endoscopic surgery. Practically, the soft endoscope is initially inserted from the mouth of the patient, the hole is made in the gastric wall, and the distal end of the endoscope is advanced through the hole into the abdominal cavity. Then, a desired procedure is performed within the abdominal cavity with a treatment instrument that is inserted into the endoscope, or with a treatment instrument that is inserted from another hole while using the endoscope as an observation device inside the abdominal cavity.

The insufflation implemented by NOTES includes insufflation for supplying a gas through an insufflation channel of an endoscope.

### SUMMARY OF THE INVENTION

An over tube according to the present invention comprises an over tube body that has an inner diameter fitting over the outer diameter of an insertion part, and is flexible and expandable in the direction of the outer diameter, a gas supply port provided on the base end side of the over tube body, an expansion/contraction part that is formed integrally with the gas supply port, provided to communicate from the base end side to a predetermined position on the distal end side, and can expand in the direction of the outer diameter by injecting a gas from the gas supply port, and at least one channel that is provided between the inner and the outer diameters of the over tube body, and penetrates from the distal end of the over tube through the base end side.

An insufflation system according to the present invention comprises an over tube body that has an inner diameter fitting over the outer diameter of an insertion part, and is flexible and expandable in the direction of the outer diameter, a gas supply port provided on the base end side of the over tube body, an insufflation device that is linked to the gas supply port, and supplies a gas to the gas supply port, an expansion/contraction part that is formed integrally with the gas supply port, provided to communicate from the base end side to a predetermined position on the distal end side, and can expand in the direction of the outer diameter by injecting a gas from the insufflation device into the gas supply port, and at least one channel that is provided between the inner and the outer diameters of the over tube body, and penetrates from the distal end of the over tube through the base end side.

In a method for using an over tube according to the present invention, the over tube comprises an over tube body that has an inner diameter fitting over the outer diameter of an insertion part, and is flexible and expandable in the direction of the outer diameter, a gas supply port provided on the base end side of the over tube body, an expansion/contraction part that is formed integrally with the gas supply port, provided to communicate from the base end side to a predetermined position on the distal end side, and can expand in the direction of the outer diameter by injecting a gas from the gas supply port, and at least one channel that is provided between the inner and the outer diameters of the over tube body, and penetrates from the distal end of the over tube through the base end side. With this configuration, the insertion part, to which the over tube with the extraction/contraction part being contracted is attached, is inserted from a natural orifice of a human being into a hole made in an organic wall, and the extraction/contraction part expands by injecting the gas from the gas supply port after the insertion part to which the over tube is attached is inserted into the hole, whereby the over tube body expands in the direction of the outer diameter, a space for forming the channel is secured, the expansion of the expansion/contraction part causes the over tube body to expand in the direction of the outer diameter, and the hole made in the organic wall is infilled.

### BRIEF DESCRIPTION OF THE DRAWINGS

FIGS. 1A and 1B show an endoscopic over tube when being contracted in a first preferred embodiment;

FIGS. 2A and 2B show the endoscopic over tube when being expanded in the first preferred embodiment;

FIGS. 3A to 3E show the structure of the endoscopic over tube in the first preferred embodiment;

FIGS. 4A to 4E exemplify the usage of the endoscopic over tube in the first preferred embodiment;

FIGS. 5A to 5B show the state where an over tube 1 is expanded in FIG. 4C;

FIGS. 6A and 6B show an endoscopic over tube 1a when being contracted in a second preferred embodiment; and

FIGS. 7A and 7B show the endoscopic over tube 1a when being expanded in the second preferred embodiment.

### DETAILED DESCRIPTION OF INVENTION

### First Preferred Embodiment

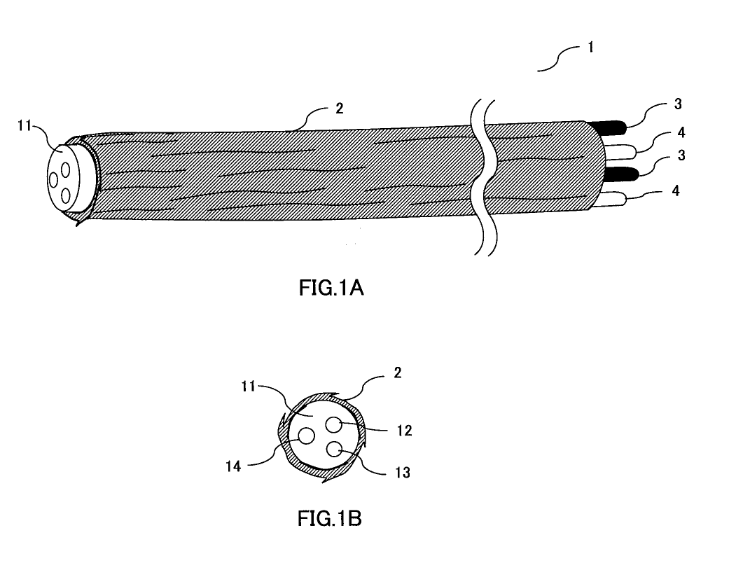

FIG. 1 shows an endoscope over tube when being contracted in this preferred embodiment. FIG. 1A is its perspective view, whereas 1B is its front view. In FIG. 1, the over tube 1 is attached to an endoscope 11 by being contracted. The distal end of the endoscope 11 includes a light guide 12, a instrument channel 13, and an air/water insufflation channel 14. Conventionally, air/water is insufflated by using the air/water insufflation channel 14. However, since the flow resistance of this channel is high due to its thinness and long length, a sufficient flow quantity cannot be secured. As a result, for example, when air is to be insufflated into the abdominal cavity, a considerable amount of time is required to expand the abdominal cavity.

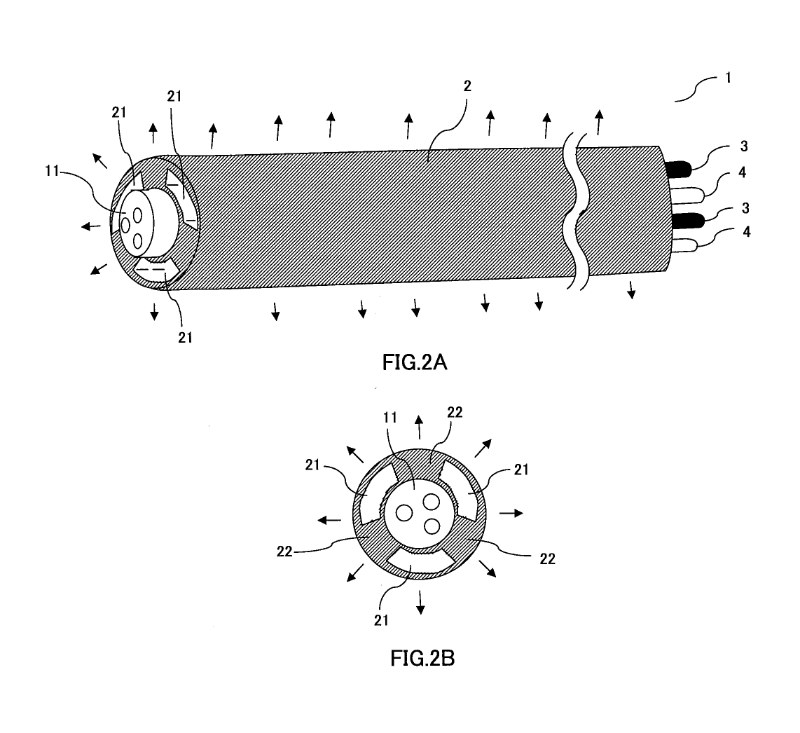

The over tube body 2 is made of a flexible material such as polyvinyl chloride, silicon rubber, etc. At the base end of the over tube body 2, expansion/contraction part control ports 3, and lumen ports 4 are provided. Each of the expansion/contraction part control ports 3 is a port for supplying a gas, or a port for suctioning a gas. By injecting a gas from the expansion/contraction part control ports 3, the over tube body 2 is expanded as shown in FIG. 2.

FIG. 2 shows the endoscope over tube when being expanded in this preferred embodiment. FIG. 2A is its perspective view, whereas FIG. 2B is its front view. By injecting a gas from the expansion/contraction part control ports 3, the expansion/contraction parts 22 are expanded. Then, lumens 21 that are deflated when being contracted emerge. The lumens 21 referred to in this preferred embodiment are channels each extending from the base end of the over tube 1 through its distal end. When a gas is injected from the lumen ports 4, it passes through the lumens 21 and is emitted from the openings of the lumens 21 at the distal end.

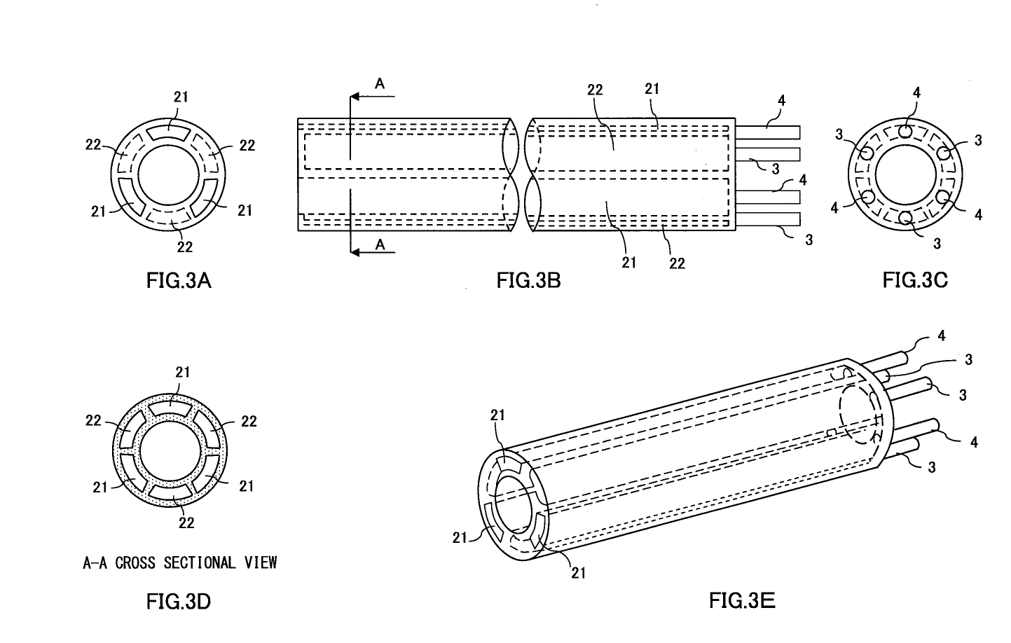

FIG. 3 shows the structure of the endoscope over tube in this preferred embodiment. FIG. 3A is its front view, FIG. 3B is its side view, FIG. 3C is its rear view, FIG. 3D is its cross-sectional view taken along a line A-A, and FIG. 3E is its perspective view.

As shown in FIG. 3, the lumens 21 and the extraction/contraction parts 22 are alternately formed within the over tube body 2. Each of the extraction/contraction parts 22 is hollow, and has a structure hermetically sealed from the outside except for the extraction/contraction part control port 3. When a gas is injected from the extraction/contraction part control ports 3, it is accumulated in the expansion/contraction parts 22, which then expand. By suctioning the gas from the expansion/contraction part control ports 3 thereafter, the gas is removed from the expansion/contraction parts 22, which are then deflated.

Each of the lumens 21 is structured to extend from the lumen port 4 through the opening at the distal end of the over tube body 2.

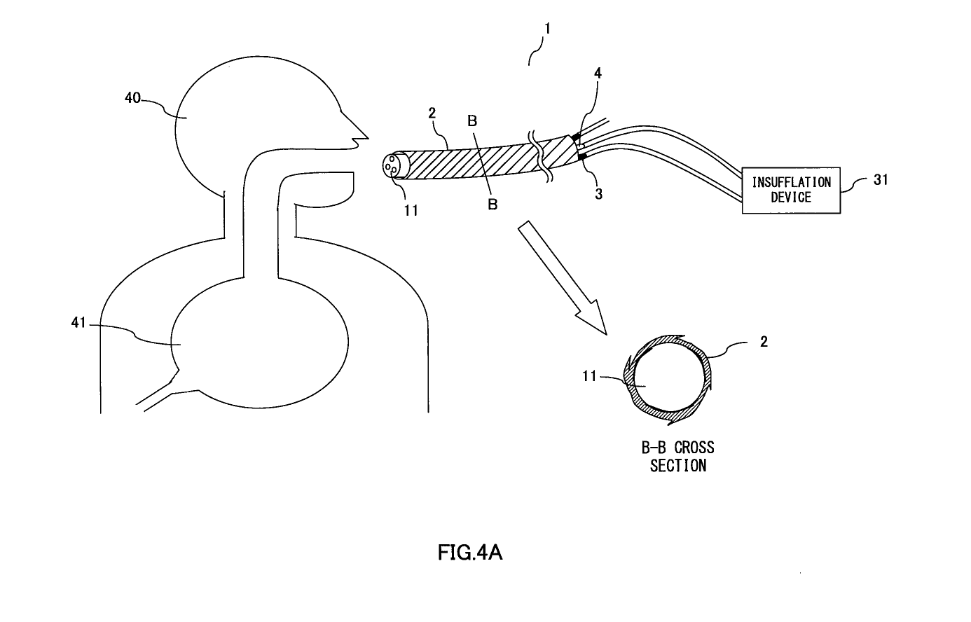

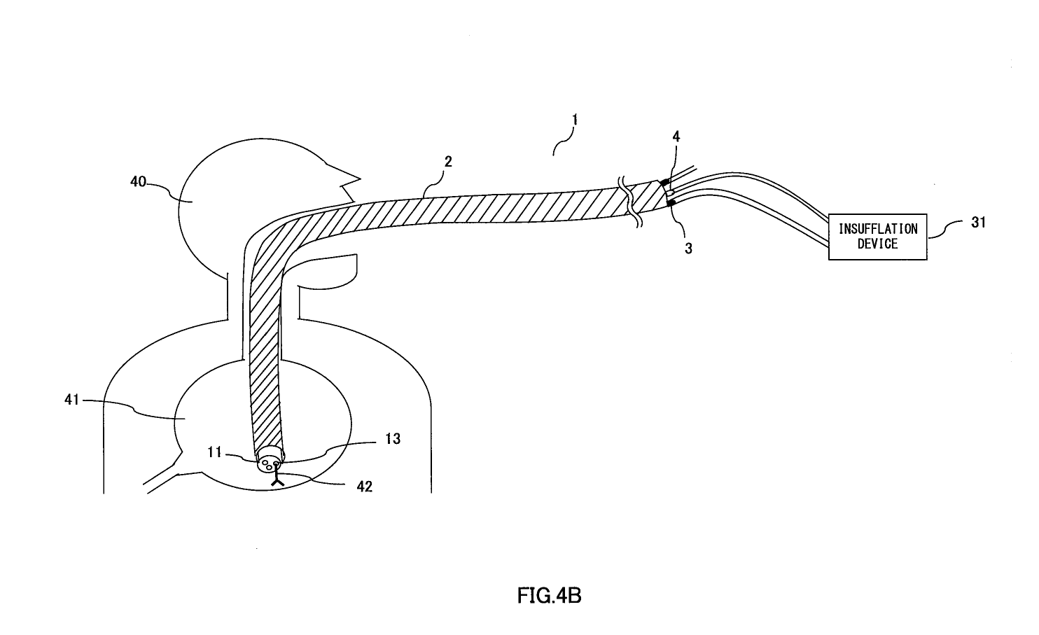

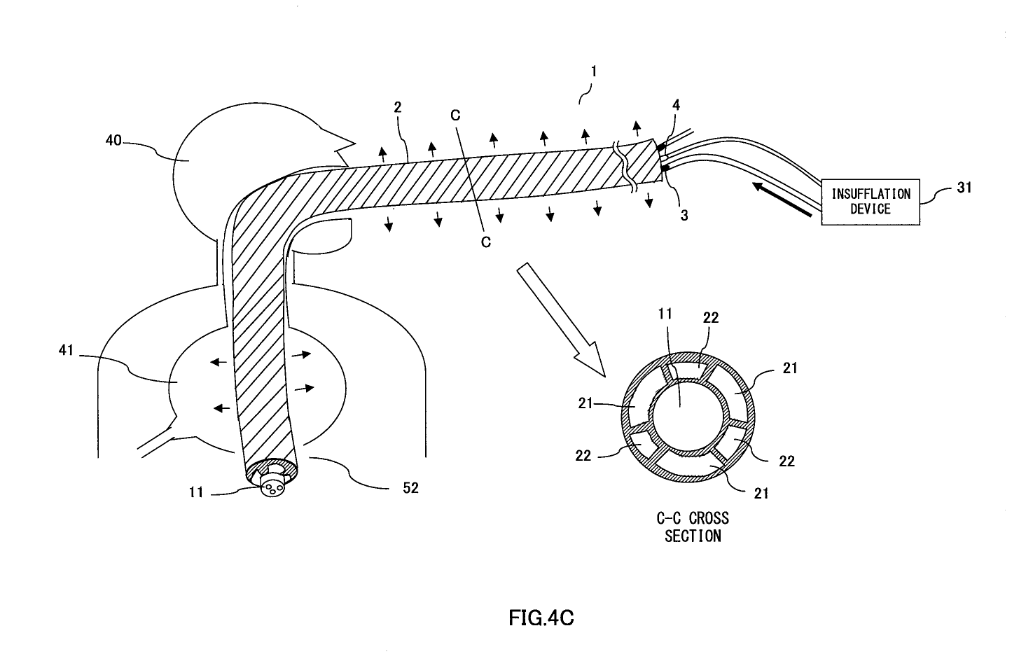

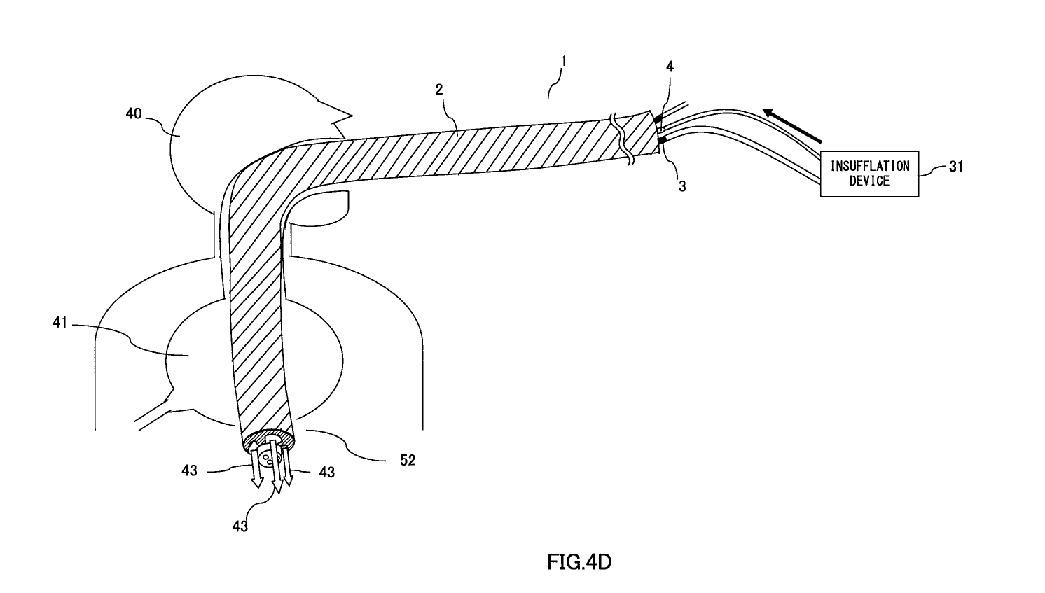

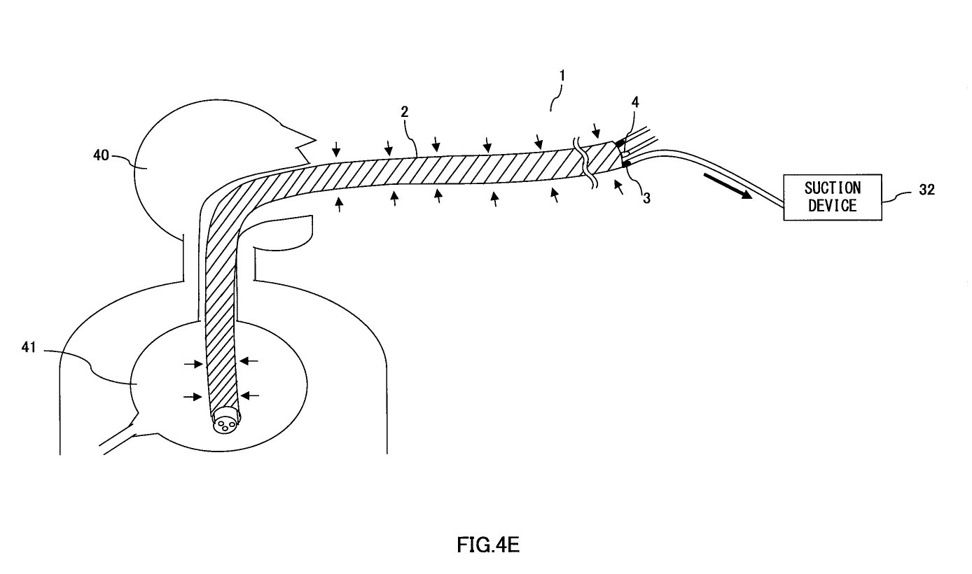

FIG. 4 exemplifies the usage of the endoscope over tube in this preferred embodiment. An example of endoscopic surgery performed with the NOTES procedure is described with reference to FIG. 4. Initially, the over tube 1 is attached to the endoscope 11. At a preceding stage where the endoscope 11 is made to reach the stomach 41 of a patient 40, the endoscope is advanced into the stomach by reducing the volume of the over tube 1 without expanding it (FIG. 4A). Namely, the expansion/contraction parts 22 are in a contracted state, and the over tube body 2 is in a deflated state.

After the endoscope 11 reaches the inside of the stomach 41, a treatment for making a hole in the gastric wall is performed to guide the endoscope 11 into the abdominal cavity (FIG. 4B). In FIG. 4B, a predetermined treatment instrument comes out of the instrument channel 13 of the endoscope 11. With this treatment instrument, the hole is made in the gastric wall.

After the hole 52 is made in the gastric wall, the endoscope 11 is inserted into the hole 52, and the distal end of the endoscope 11 is made to reach a lesion portion. Then, an insufflation device 31 is operated to inject a gas from the expansion/contraction part control ports 3. Then, the gas injected from the expansion/contraction part control ports 3 is accumulated in the expansion/contraction parts 22, which then expand (FIG. 4C). As a result, the over tube body 2 expands, and the lumens 21 that are deflated when being contracted are formed.

After forming the lumens 21, the insufflation device 31 is operated to inject a gas from the lumen ports 4. The gas 43 injected from the lumen ports 4 passes through the lumens 21 within the over tube body 2, and is emitted from the openings at the distal end of the over tube body 2 (FIG. 4D). Then, the gas 43 is insufflated into the abdominal cavity (FIG. 4D). As a result, a sufficient flow quantity can be obtained because the insufflation channels are thicker than the air/water insufflation channel.

After the lesion is treated and the gastric wall is sutured with the endoscope 11, a suction device 32 is operated to suction the gas from the expansion/contraction part control ports 3 in order to reduce the volume of the over tube body 2. Then, the gas accumulated in the expansion/contraction parts 22 is suctioned from the expansion/contraction part control ports 3, the expansion/contraction parts 22 are contracted, thereby the over tube body 2 is contracted (FIG. 4E). Thereafter, the endoscope 11 is drawn out of the stomach 41 of the patient 40.

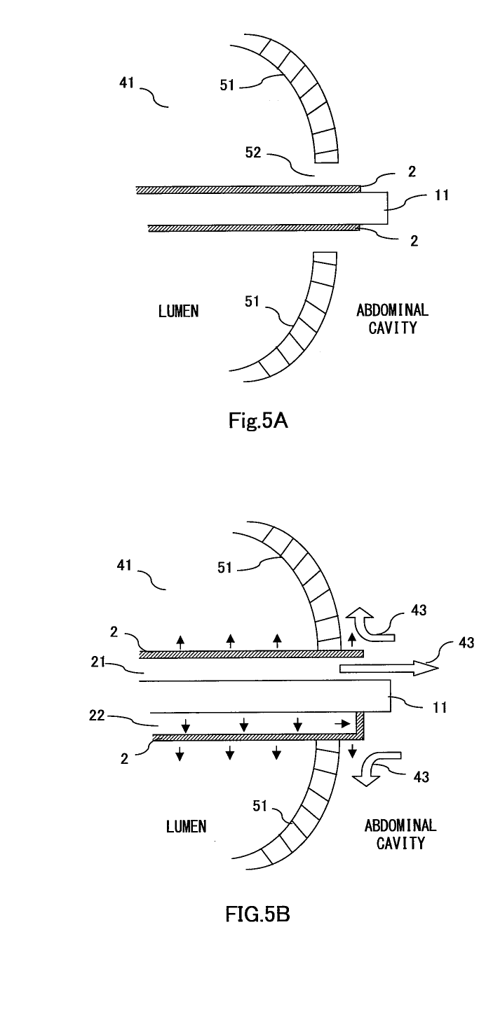

FIG. 5 shows the state where the over tube 1 is expanded in FIG. 4C. After the hole 52 is made in the gastric wall 51 in FIG. 4B, the endoscope 11 is inserted into the hole 52 as shown in FIG. 4C. The state when viewed in the direction of the side at this time is shown in FIG. 5A.

Thereafter, by injecting the gas from the expansion/contraction part control port 3, the expansion/contraction parts 22 expand as shown in FIG. 5B, thereby the over tube body 2 expands. At this time, the over tube body 2 expands and completely infills the hole 52. In this way, the gas 43 that is insufflated into the abdominal cavity through the lumens 21 can be prevented from leaking into the stomach 41 through the hole 52.

The gas 43 is supplied by using the lumens 21 as described above. However, a liquid such as water, etc. may be injected from the lumen ports 4, and emitted from the openings at the distal end of the over tube body 2. Additionally, the suction device 32 may be linked to the lumen ports 4 to suck the liquid through the lumens 21. Furthermore, the lumens may be assigned in such a way that one is for supplying a gas, one is for supplying a liquid, and another is for suction. Still further, the number of lumens is not limited to three, and may be one or more. Still further, the expansion/contraction parts 22 may be linked. In this case, the number of expansion/contraction part control ports 3 can be one.

### Second Preferred Embodiment

An endoscope over tube implemented by sheathing the over tube 1 in the first preferred embodiment with an elastic cover is described next.

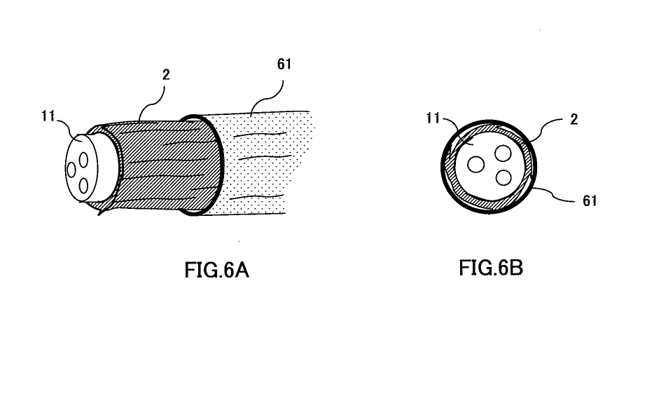

FIG. 6 shows the endoscope over tube 1a when being contracted in this preferred embodiment. FIG. 6A is its perspective view, whereas FIG. 6B is its front view. In FIG. 6, the over tube body 2 shown in FIG. 1 is sheathed with an elastic material (cover 61) such as rubber, nylon, etc. The cover 61 fastens the over tube body 2 when the expansion/contraction parts 22 are not filled with a gas. Therefore, the outer diameter of the over tube 2 can be made smaller than that shown in FIG. 1.

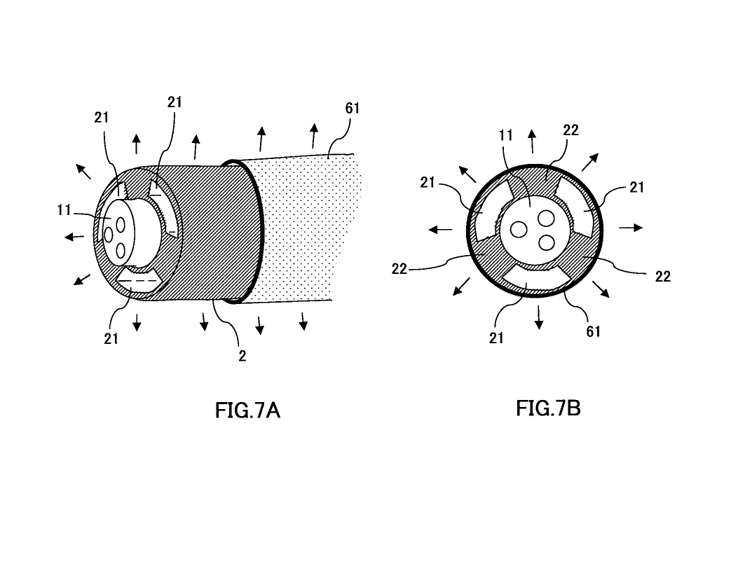

FIG. 7 shows the endoscope over tube 1a when being expanded in this preferred embodiment. FIG. 7A is its perspective view, whereas FIG. 7B is its front view. By injecting a gas from the extraction/contraction part control ports 3, the expansion/contraction parts 22 expand, thereby the over tube body 2 expands. At the same time, also the cover 61 expands due to its elasticity. Then, the lumens 21 are formed in a similar manner as in the first preferred embodiment.

According to this preferred embodiment, the cover 61 fastens the over tube body 2 by sheathing the over tube body 2 with the cover 61, whereby the outer diameter of the over tube body 2 can be made smaller than that shown in FIG. 1. This makes it easier to insert the endoscope 11 into the esophagus and the stomach of a patient. Additionally, this can relive the discomfort caused when ripples (wrinkles), which are generated by the contraction of the over tube body 2 resultant from the suction of the gas of the expansion/contraction parts 22 from the expansion/contraction part control ports 3 of the over tube 1, pass through the esophagus, etc. of a patient.

As described above, the endoscope over tubes according to the preferred embodiments of the present invention comprise an over tube body and gas supply ports. The over tube body has the inner diameter fitting over the outer diameter of the endoscope, and is flexible and expandable in the direction of the outer diameter. The gas supply ports are provided on the side of the base end of the over tube body in order to insufflate a gas into the over tube body. Additionally, at least one channel is extended from the distal end of the over tube through its base end within the fringe portion between the inner and the outer diameters of the over tube body. The inside of the over tube body has hollow spaces, and hermetically sealed from the outside except for the gas supply ports.

With this configuration, the hollow spaces expand by injecting a gas into the over tube body from the gas supply ports when the hollow spaces are contracted, thus expanding the over tube body in the direction of the outer diameter. As a result, the space for implementing the channel can be secured.

Additionally, a plurality of the above described channels may be comprised. Such a configuration enables the channels to be used in such a way, for example, one channel is for supplying a gas, one channel is for supplying a liquid, and another channel is for suction.

The endoscope over tube may further comprise an elastic cover that sheathes the outer surface of the over tube body. Such a configuration causes the cover 61 to fasten the over tube body 2, whereby the outer diameter of the over tube body can be further reduced when the endoscope is inserted into the body.

The over tube according to the present invention includes the over tube body, the gas supply ports, the expansion/contraction parts, and at least one channel as described above. The over tube body has the inner diameter fitting over the outer diameter of the insertion part, and is expandable in the direction of the outer diameter. The gas supply ports are provided on the side of the base end of the over tube body. The expansion/contraction parts are formed integrally with the gas supply ports, and provided to communicate from the base end side through a predetermined position on the distal end. By injecting a gas from the gas supply ports, the expansion/contraction parts can expand in the direction of the outer diameter. The channels are provided between the inner and the outer diameters of the over tube body, and penetrate from the distal end side of the over tube to the base end side.

It is to be understood that the above described insertion part is not limited to the soft endoscope referred to in the above described preferred embodiments. The insertion part includes, for example, an insertion part of a surgical endoscope, or a medial treatment instrument such as a trocar, etc. By applying the present invention to an industrial endoscope, for example, an insufflation destination can be cleaned by insufflation, and a field of view can be secured.

According to the present invention, the endoscope is guided into the body when the diameter of the over tube is reduced by contracting the endoscope over tube, the insufflation channel that can supply a sufficient flow quantity can be secured by expanding the endoscope over tube at the time of a treatment, and the over tube can be again contracted when being drawn out of the body of a patient. Accordingly, the burden on the patient when the endoscope is inserted into the body can be reduced, and at the same time, the insufflation channel that can supply a flow quantity sufficient for a treatment can be secured.

It is to be understood that the present invention is not limited to the above described preferred embodiments, and various configurations or embodiments can be implemented within the scope that does not depart from the spirit of the present invention.

What is claimed is:

1. An over tube, comprising:
an over tube body that has an inner diameter fitting over an outer diameter of an insertion part, and is flexible and expandable in a direction of the outer diameter;
a gas supply port provided on a base end side of said over tube body;
an expansion/contraction part that is formed integrally with said gas supply port, provided to communicate from the base end side to a predetermined position on the distal end side, and can expand in the direction of the outer diameter by injecting a gas from said gas supply port; and
at least one channel that is provided between the inner and outer diameters of said over tube body, and penetrates from the distal end of the over tube through the base end side.

2. The over tube according to claim 1, wherein
said expansion/contraction part expands by injecting a gas from said gas supply port into said expansion/contraction part when said expansion/contraction part is contracted, whereby said over tube body expands in the direction of the outer diameter, and a space for forming said channel is secured.

3. The over tube according to claim 1, wherein
there are a plurality of said channels.

4. The over tube according to claim 1, wherein
said over tube further comprises an elastic cover for sheathing an outer surface of said over tube body.

5. An insufflation system, comprising:
an over tube body that has an inner diameter fitting over an outer diameter of an insertion part, and is flexible and expandable in a direction of the outer diameter;
a gas supply port provided on a base end side of said over tube body;
an insufflation device that is linked to said gas supply port, and supplies a gas to said gas supply port;
an expansion/contraction part that is formed integrally with said gas supply port, provided to communicate from the base end side to a predetermined position on a distal end side, and can expand in the direction of the outer diameter by injecting a gas from said insufflation device into said gas supply port; and
at least one channel that is provided between the inner and outer diameters of said over tube body, and penetrates from the distal end side of the over tube through the base end side.

6. The insufflation system according to claim 5, wherein
said insufflation device is further linked to an opening of said channel on the base end side of the over tube, and supplies a gas to said channel.

7. A method for using an over tube, wherein:
said over tube comprises
an over tube body that has an inner diameter fitting over an outer diameter of an insertion part, and is flexible and expandable in a direction of the outer diameter,
a gas supply port provided on a base end side of said over tube body,
an expansion/contraction part that is formed integrally with said gas supply port, provided to communicate from the base end side to a predetermined position on a distal end, and can expand in the direction of the outer diameter by injecting a gas from said gas supply port, and
at least one channel that is provided between the inner and outer diameters of said over tube body, and penetrates from the distal end of said over tube through the base end side, wherein;
the insertion part, to which the over tube with said extraction/contraction part being contracted is attached, is inserted from a natural orifice of a human being into a hole made in an organic wall; and
said extraction/contraction part expands by injecting the gas from said gas supply port after the insertion part to which the over tube is attached is inserted into the hole, and said over tube body expands in the direction of the outer diameter, whereby a space for forming said channel is secured, and because the expansion of said expansion/contraction part causes said over tube body to expand in the direction of the outer diameter, the hole made in the organic wall is infilled.
